(19) United States
(12) Patent Application Publication
(10) Pub. No.: US 2025/0341456 A1
(43) Pub. Date: Nov. 6, 2025

(54) SYSTEMS AND METHODS FOR CONTROLLING AND ANALYZING TEMPORAL DYNAMICS IN SINGLE CELLS AND CELL POPULATIONS

(71) Applicant: Vanderbilt University, Nashville, TN (US)

(72) Inventors: Gregor Neuert, Nashville, TN (US); Alexander Thiemicke, Nashville, TN (US); Abraheem Khouqeer, Nashville, TN (US); Hossein Jashnsaz, Nashville, TN (US); Jason J. Hughes, Nashville, TN (US)

(21) Appl. No.: 18/864,471

(22) PCT Filed: May 15, 2023
(86) PCT No.: PCT/US2023/066991

§ 371 Date: Nov. 8, 2024

Related U.S. Application Data
(60) Provisional application No. 63/341,509, filed on May 13, 2022.

(51) Int. Cl. G01N 15/14; C12M 1/00; C12M 1/36; G01N 15/10

(52) U.S. Cl. CPC G01N 15/1459; C12M 29/00; C12M 41/14; C12M 41/48; G01N 2015/1006

(57) ABSTRACT

Systems and methods for gradient profile generation and automated cell cytometry analysis are disclosed. The technology comprises a computer controller, one or more syringe pumps, an incubator comprised of one or more cell cultures, an autosampler connected to a peristaltic pump, and a multi-line switch valve in communication with the incubator. The multi-line switch valve is configured to add one or more of media, PBS, quenching reagent, or inactivation reagent to the one or more cell cultures. The computer controller mediates the injection of stimulus to the cell cultures via the one or more syringe pumps based on a computed temporal pump profile that determines the stimulus concentration over discrete time points.

### CROSS-REFERENCE TO RELATED APPLICATIONS

This application claims the benefit of U.S. Provisional App. No. 63/341,509, filed May 13, 2022, the entire contents of which are incorporated herein by reference.

### FIELD OF THE INVENTION

The present invention is directed to novel systems and methods of gradient profile generation of arbitrary type and the manipulation of cells with these profiles as well as automated flow cytometry analysis of these cells.

### BACKGROUND OF THE INVENTION

Dynamic changes in cellular stimuli within an organism can lead to variations in cell responses including protein signaling over time. To understand these changes in a controlled cell culture environment, it is necessary to generate physiologically relevant environments and analyze the dynamics of cell responses including protein signaling simultaneously and over time. Most current cell culture experiments are performed in acute or constant environmental changes that do not consider the dynamics of physiological environments of cells as observed for example in a human or an animal. In addition, analysis of these perturbed cells is often performed without robust time series analysis. Flow cytometry stands out as a broadly applicable single-cell technique that enables high throughput time-course experiments and efficient data acquisition of millions of cells. Multiple parameters of morphology, protein expression and phosphorylation per cell can be measured via conventional flow cytometry. Fluorescent cell barcoding (FCB) is an emerging flow cytometry approach that utilizes covalently bound fluorescent dyes to stain cells with discrete fluorescence intensities. Barcoded samples of different time points can be pooled and stained with antibodies of interest. Processing pooled barcoded samples in one tube results in high-throughput data acquisition and reduced experimental variation in antibody staining compared to conventional flow cytometry.

Although a variety of flow cytometry software analysis methods exist that allow for manual gating and semi-automated analysis, their limitations lie in the subjective nature of human biased analysis that can be difficult to reproduce. Additionally, batch analysis of multiple experiments using conventional software, can be time consuming as it requires manual adjustments of gating strategies for each experiment due to antibody staining variations. For time-course and multiplexed experiments, the data analysis becomes even more tedious and labor-intensive as it requires an extra gating strategy to demultiplex the barcoded samples.

### SUMMARY OF THE INVENTION

To address those and other deficiencies in the art, described herein are systems and methods to generate profiles of molecules that change over time. These molecules can be biological or chemical in nature to perturb biological cells. These profiles can increase or decrease over time in any linear or non-linear form. The goal is to perturb cells with these gradual profiles to elicit a cellular repones. The profiles are generated by using a computer programmable pump that inject a high concentration solution of the molecule into a flask or beaker with growth media under constant mixing through a stir bar, a shaker, or a vortex shaker. In the first setup, a flask contains growth media with cells. These cells are exposed to temporal changes in the environment of the molecule. Samples of cells are collected at predefined time points. In the second setup, the beaker contains media without cells. In this setup a second pump draws liquid from the beaker and runs it through a flow chamber that contains cells. As the first pump inject high concentration of the molecule to the flask or the beaker, the concentration of the molecule increases with the rate at which the pump injects the molecule. To account for concentration changes in the flask or beaker due to sampling or media withdraw, the pump rate is adjusted in predetermined time points.

The technology of the present invention can be used to generate any type of increasing or decreasing concentration of molecules to study cells response to biomolecules, chemicals or drugs. Alternative approaches use gravity-based flow systems or microfluidic systems. Among the advantages of the present technology over the gravity approach is that it can generate a wide variety of flow rates and therefor concentration profiles. An advantage over microfluidic systems is that the present technology does not require microfabrication. In addition, the present technology is also compatible with a wide variety of standard molecular, cell biology, or genomic assays currently available.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a diagram of an exemplary embodiment of the hardware of the system of the present invention in an implementation for gradient generation and automated sampling for TP (time-point) experiments;

FIG. 2 is a diagram showing an exemplary process for computing temporal pump profiles;

FIGS. 3A-3J show graphs related to example pump profile calculation generated for a TP (time-point) experimental setup as shown in FIG. 1 for a linear NaCl concentration increase to 0.150M over 600 minutes;

FIG. 4 is a diagram of an exemplary embodiment of the hardware of the present invention in an implementation for a gradient profiler for flow systems for TS (time-series) experiments;

FIG. 5A-5J show graphs related to an example pump profile calculation generated for a TS (time-series) experimental setup as shown in FIG. 4 for a quadratic NaCl concentration increase to 0.4M over 25 minutes;

FIG. 6 is a diagram of an exemplary embodiment of the hardware of the present invention in an implementation for adherent cells in multiwall plates; and

FIG. 7 is a diagram of an exemplary embodiment of the hardware of the present invention in an implementation using stackable flasks for adherent cells.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

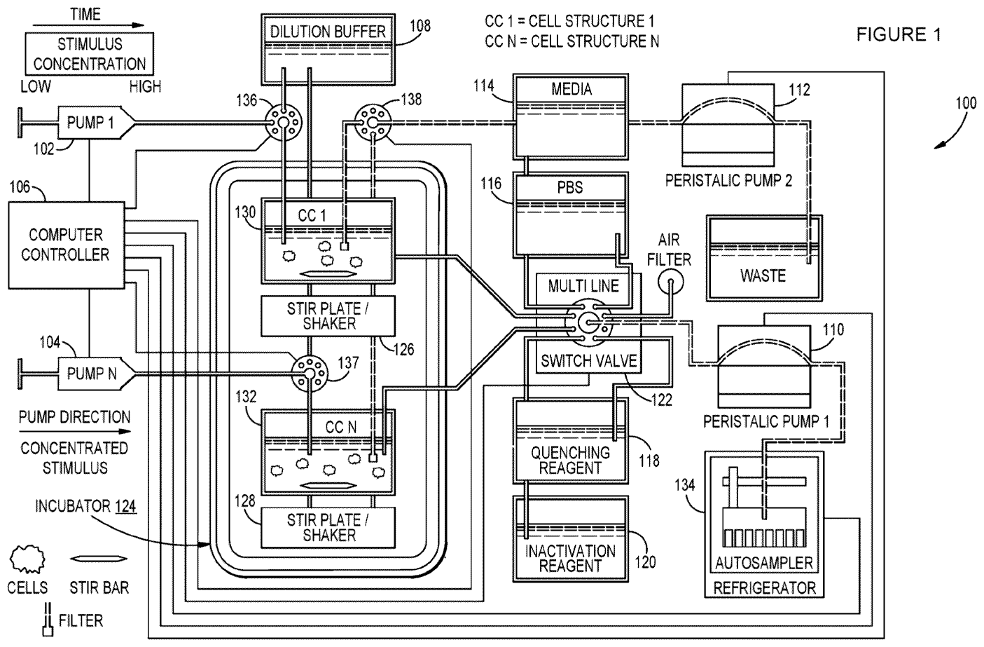

FIG. 1 is a diagram of an exemplary embodiment of the hardware of the system 100 of the present invention in an implementation for gradient generation and automated sampling. The system 100 is comprised of one or more syringe pumps 102, 104, a computer controller 106, and a cell culturing incubator 124. The incubator is further comprised of one or more stir plates or shaker platforms 126, 128, and one or more cell cultures 130, 132 that are suspended in appropriate media. Multi-line switch valves 122, 136, 138 connect the dilution media 108 to the cell culture 130, 132 and the waste collection through the peristaltic pump 112. Media 114, PBS 116, quenching reagent 118, and inactivation reagent 120 connect the autosampler 134 through the peristaltic pump 110 to the multi-line switch valve 122.

The computer controller 106 is comprised of a central processing unit, a storage medium, a user-input device, and a display. Examples of computers that may be used are: commercially available personal computers, open source computing devices (e.g. Raspberry Pi), commercially available servers, and commercially available portable devices (e.g. smartphones, smartwatches, tablets, laptops).

Over time, as shown in FIG. 1, the pumps 102, 104 are responsible for increasing the stimulus concentration to the cell cultures 130, 132 from a lower concentration to a higher concentration. The computer controller 106 controls the automated operation of the system through valve, including a multi-line switch valves 122, 136, 138, that control the flow of dilution buffer 108, and the stimulus through the syringe pumps 102, 104 into the cell cultures 130, 132. The computer controller 106 also controls a first peristaltic pump 110, and second peristaltic pump 112, and the autosampler 134 that can be refrigerated. Through the first peristaltic pump 110, the computer controller 106 controls the sampling performed by the autosampler 134 at predetermined time periods from the cell cultures 130, 132 through the multi-line switch valve 122. Through the second peristaltic pump 112 and the multi-line switch valves 136, 138, the computer controller 106 controls the addition of dilution buffer 108 to reduce the stimulus concentration in the cell cultures 130, 132 and extract waste from the cell cultures 130, 132. In certain embodiments, the computer controller 106 may be remote from the rest of the system of the present invention and in communication to the various components via a network.

Media 114, PBS 116, quenching reagent 118, and inactivation reagent 120 are transported through the multi-line switch valve 122 and the peristaltic pump 110 to the autosampler 134 which are all controlled by the computer controller 106. The computer controller also operates the air filter 114 system through the multi-line switch valve 122.

The computer controller 106 uses the pumps 102, 104 to add a defined volume of concentrated stimulus to the cell cultures 130, 132 over a predetermined period of time. The concentration can be altered by introducing dilution buffer 108, applying stimulus to the cell cultures 130, 132, and utilizing controlled multi-switch valves 136, 138 along with peristaltic pump 112 to remove specific volumes to the waste container. Those changes in the pump profile calculation are accounted for as follows. During each interval, stimulus over time is delivered continually by adding appropriate amount (dvᵢ) of concentrated stimulus (Cₘₐₓ) to the total volume of growth media in a cell culture 130, 132.

The computer controller 106 communicates with each of components through a communication module/APIs directly or through a controller board. The user-defined inputs provided to the computer controller 106 include: (1) the number of experiments; (2) the desired timepoints to sample; (3) the desired time delay to add a quenching or inactivation reagent; (4) the number of tubes per timepoint to sample; and (5) the sampling and washing volumes.

Typically, in operation, the computer controller will prepare the experiment itself, by priming the tubing connecting it to the other components of the system, including the media 114, PBS 116, quenching reagent 118, and inactivation reagent 120. Based on the foregoing input parameters, the computer controller computes and optimizes the timing for turning the peristaltic pumps 110 on and off to sample cells of a predetermined volume from cell culture flasks 130, 132. The computer controller 106 also computes and optimizes the timing and switching of the multi line switch valves 136, 138 and turning the peristaltic pump 112 on and off to dilute the stimulus concentration with the dilution buffer 108. The computer controller 106 also determines when to activate and where to position the autosampler 134 for sample collection, when to quench or inactivate the sample with a predetermined volume using the quenching reagent 118 or inactivation reagent 120, and when to execute a PBS 116 washing step after each time point followed by removal of PBS into the waste using filtered air 114. In certain embodiments, the minimum time between time points is thirty seconds.

After the samples have been collected by the autosampler 134, flow cytometry may be used to acquire cell parameters at each sampled timepoint. Flow cytometry analysis is automated using a custom software running on the computer controller 106, which is capable of reading flow cytometry standard (FCS) files obtained from any conventional flow cytometry instrument. At the computer controller 106, the user may input the number of replica and their number of time points and corresponding barcoding dye intensities (up to 3 dyes and 3 to 4 concentrations per dye); the fluorescent channels of the barcoding dyes, the forward and side scatter channel and a predefined specific molecular marker channel, and the cluster density standard deviation for all channels.

Using those inputs, the software running on the computer controller 106 performs automated doublet discrimination. The cell population selection is based on forward and side scatter and a predefined specific molecular marker based on input parameters. The remaining cells are demultiplexed for replica experiments. For each replica experiment, the computer controller demultiplexes the user defined timepoints which is based on the different barcoding dyes and their intensities. The software then visualizes the fluorescent distributions of the predefined channels as a ridge line plot. The software then combines individual replica files by the median and the standard deviation of the replica distributions and performs a comparison of different experimental conditions for the same molecular marker. The software then performs a normalization of different molecular markers by using the unstained cell populations to determine relative changes and uses the single cell distributions to compute summary statistics such as the percent positive or the distribution median of the parameter of interest. The software also performs automated statistical testing of time course data sets from different replica experiments, applying different experimental conditions and different molecular markers of interest. The software can then prepare a report containing all analyses, which may be saved for each flow cytometry file. Multiple files can be analyzed simultaneously and in parallel using the software running on the computer controller 106.

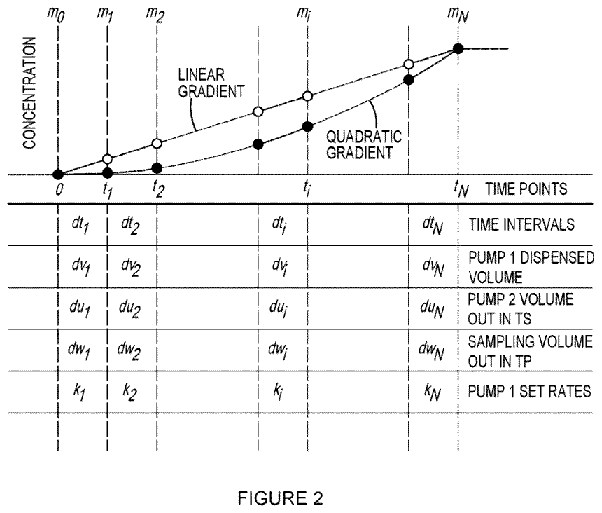

The system of the present invention computes temporal pump profiles in order to determine the stimulus concentration for any profile over discrete time points, which are set by the programmable syringe pumps 102, 104 and peristaltic pumps 110, 112 by combining several short segments with linear concentration profile. An exemplary representation of calculating temporal pump profiles is shown in FIG. 2.

A desired concentration profile consists of a maximum number of discrete time intervals set by the number of phases a programmable syringe pump provides. One may construct any arbitrarily time-varying concentration profile by combining short segments with linear concentration changes. During each time interval, the concentration is increased linearly with a fixed rate drᵢ. By changing the rate from one interval to the next, any arbitrary temporal profile may be produced over the whole treatment time.

A process to automatically account for changes to concentration in the cell cultures and compute pump profiles is outlined below:
Time points are defined as: [t₁, t₂, t₃, . . . t_{N}]
Time intervals between time points are defined as: [dt₁, dt₂, dt₃, . . . dt_{N}]
The desired stimulus concentration at time point tᵢ is defined as: mᵢ
The fixed volume of concentrated stimulus from syringe pumps 102, 104, 404 to be added to the mixing flasks 130, 132, 420 during interval dtᵢ is defined as: dvᵢ
At the rate kᵢ of syringe pumps 102, 104, 404, the dvᵢ volume of concentrated stimulus is added to the mixing flasks 130, 132, 420 during interval dtᵢ and is calculated as: kᵢ=dvᵢ/dtᵢ.
The mixing flasks 130, 132, 420 have an initial volume of V₀ and an initial stimulus concentration of m₀=0 at t=0.
The stimulus concentration profile at any given time point (mᵢ) is then calculated as:

$${m}_{i} = {m}_{i - 1} + \frac{{C}_{ma x} {dv}_{i} - \overline{m} ( {du}_{i} + {dw}_{i} )}{{V}_{i} + {dv}_{i} - ( {du}_{i} + {dw}_{i} )}$$

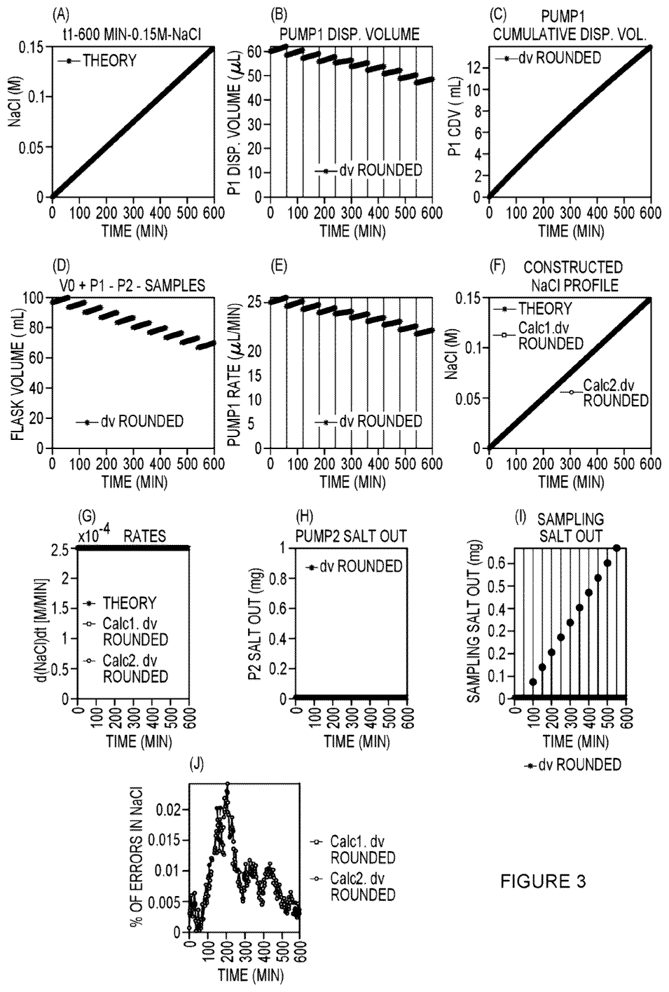

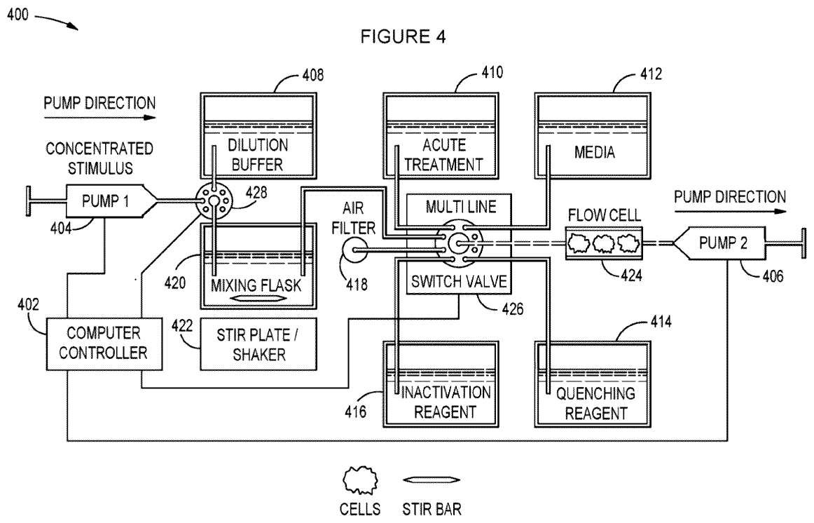

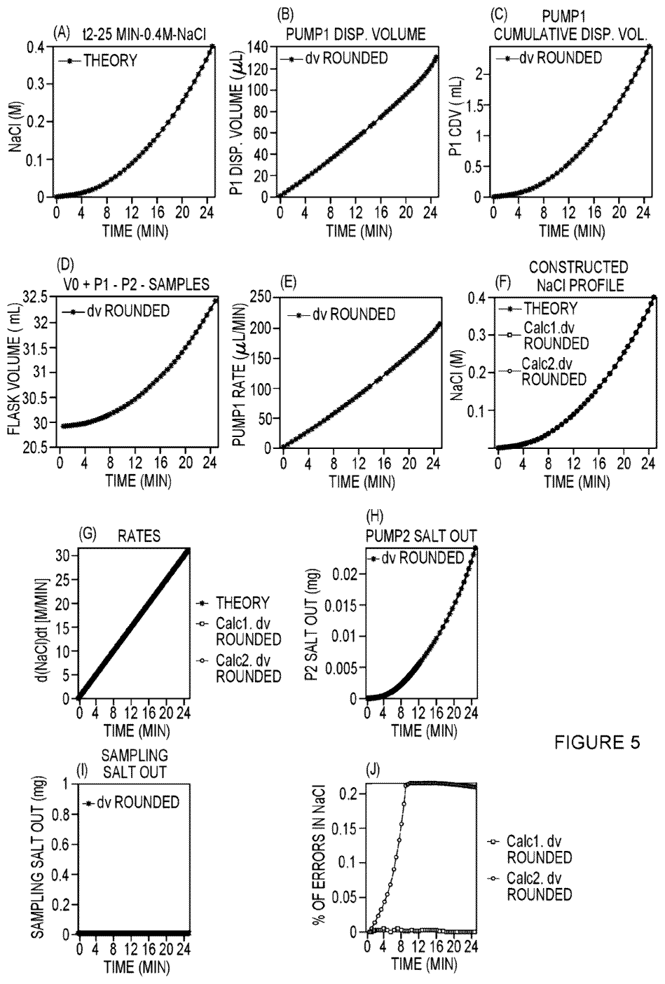

Cₘₐₓ is the concentrated stimulus (in the unites of mM) loaded to the first syringe pumps 102, 104, 404
m̅ is the average of mᵢ and mᵢ₋₁ (in the unites of mM)
dvᵢ (in mL) is the dispensed volume of concentrated stimulus through syringe pumps 102, 104, 404 during the time interval dtᵢ.
duᵢ (in mL) is the volume taken out by the second syringe pump 406 (in TS experiment, as outlined in FIG. 5, and implemented in FIG. 4) during the interval dtᵢ
dwᵢ (in mL) is the volume taken out by sampling (in TP experiments, as outlined in FIG. 3, and implemented in FIG. 1) with a second peristaltic pump 110 or taken out by peristaltic pump 112 for diluting concentration in cell culture flasks 130, 132 with dilution buffer 108 during the interval dtᵢ.
Vᵢ is the total flask volume of mixing flasks 130, 132, 420 (in mL) at tᵢ.
Once dvᵢ is computed, then the pump rate is computed as kᵢ=1000*dvᵢ/dtᵢ in μL/min.
For time series experiments (as outlined in FIG. 5) and TP experiment (as outlined in FIG. 3), the second pumps 110, 112, 406 are operated at a rate of k̅, thus duᵢ=k̅+dtᵢ.
The calculated values of dvᵢ are rounded in the specified unit to 3 digits after the decimal which is the functional value for the syringe pumps 102, 104, 404, 406 and the peristaltic pumps 110, 112.

FIG. 3 is an example pump profile calculation generated for a TP (time-point) experimental setup as described in FIG. 1 for a linear NaCl concentration increase to 0.150M over 600 minutes. As shown in FIG. 3A, the above outlined process is applied to increase the stimulus—NaCl—in concentration in a linear fashion over a time period. In these time point experiments, during each time interval, a defined volume P1 of concentrated stimulus is added to the mixing flasks 130, 132 using a syringe pumps 102, 104 (FIG. 3B). FIG. 3C describes the accumulated dispensed volume of P1 over time. FIG. 3D describes the volume in the mixing flask 130, 132, which is the sum of the initial volume V0, the added volume P1, minus the volume P2 taken out by peristaltic pump 110 and 112 and which is 0 in this example, and minus the sampling volume Samples. FIG. 3E describes the pump rate over time of syringe pumps 102, 104. As shown in the FIG. 3F, through the application of the above calculations, an NaCl pump profile can be designed to match closely to the theoretical increase over the 600 minute time period. FIG. 3G plots the rate of NaCl change over time for the theoretical value and through the application of the above calculations. FIG. 3H displays the rate of peristaltic pumps 110, 112. At each time intervals, a fixed volume is taken out of the flask using the hardware setup described in FIG. 1 of the desired concentration of the stimulus (FIG. 3I). As shown in FIG. 3F, through the application of the above calculations, an NaCl pump profile can be designed to match closely to the theoretical increase over the 600 minute time period with a % error shown in FIG. 3J.

FIG. 4 is a hardware design used to generate the profiles in FIG. 5 of the present invention in an implementation for a gradient profiler for flow systems, where the system monitors the effluent from a gradient so it can locate certain particles in the fractions created during the run. In the system 400, the computer controller 402 controls a first syringe pump 404, second syringe pump 406, the mixing flask 420, the stir plate or shaker 422, and the multi-line switch valves 426, 428. Through the multi-line switch valve 428, the computer controller 402 controls the addition of concentrated stimulus in syringe pumps 404, 406 to the mixing flask 420 to increase the concentration. The dilution buffer 408 through the multi-line switch valves 428, and the syringe pump 406 dilutes the concentration in the mixing flask 420. Through the multi-line switch valve 426, the computer controller 402 controls the addition of acute treatment 410, the addition of media 412, the addition of the quenching agent 414, and the addition of the inactivation agent 416 to the flow cell 424. The computer controller also operates the air filter 418 system through the multi-line switch valve 426. The multi-line switch valve 426 and a second syringe pump 406 is used to add a flow of cells to the flow cell 424 and then deliver solution from the mixing flask 420 in order to perform the gradient profiling. In certain embodiments, the computer controller 402 may be remote from the rest of the system of the present invention and in communication to the various components via a network.

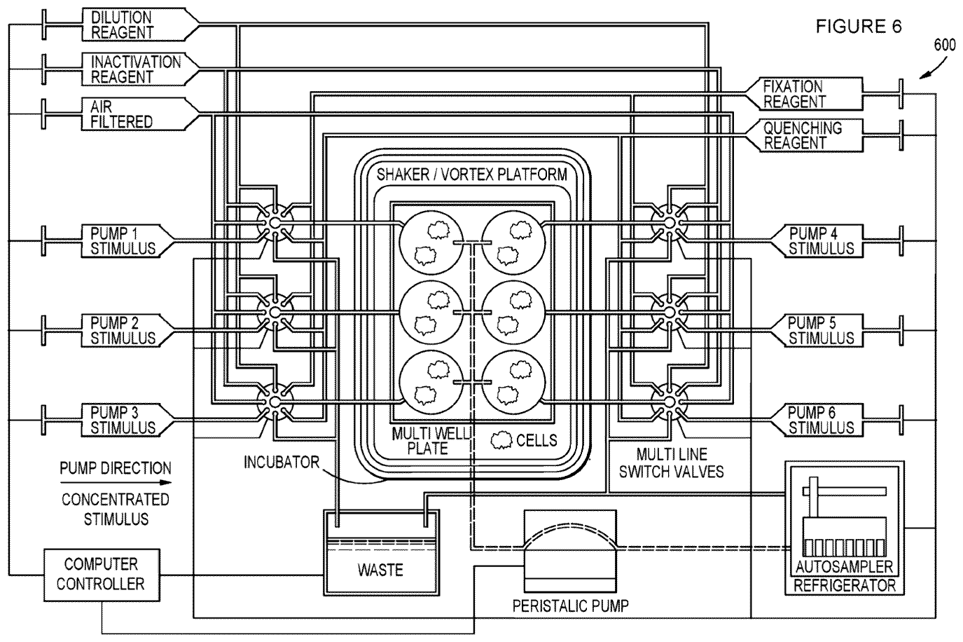

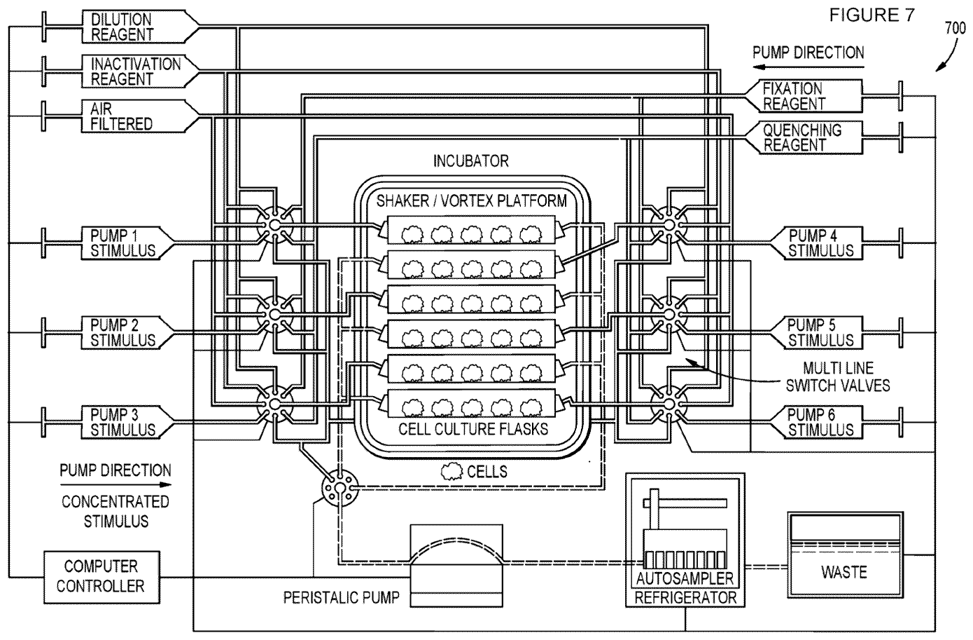

FIG. 5 is an example pump profile calculation generated for a TS (time-series) experimental setup as described in FIG. 4 for a non-linear (quadratic) NaCl concentration increase to 0.4M over 25 minutes (FIG. 5A). The total dispensed volume P1 by syringe pump 404 is plotted in FIG. 5B and the cumulative total dispensed volume P1 is plotted in FIG. 5C. In FIG. 5D, the volume in the mixing flask 420 consist of the initial volume V0, the added volume P1 through the syringe pump 404, minus the volume P2 taken out by the syringe pump 406, and minus the sampling volume Samples which is 0 in this application. The rate of the syringe pump 404 is shown in FIG. 5E. A time series experiment performs successive measurements from the same source such as cells in a flow cell 424 over a fixed time interval and is used to track change over time (FIG. 5F). The calculated concentration (FIG. 5F) and rates (FIG. 5G) changes match the predicted concentration and rate changes. In time series (TS) experiments, the second pump 406 pulls media at a fixed rate (FIG. 5H) from the mixing flask 420 and though the flow cell 424 resulting in a predefined stimulus concentration to cells in a flow cell. The sampling volume is 0 (FIG. 5I). The % error in the calculated concentration change plotted in FIG. 5J. FIGS. 6 and 7 similarly show alternative embodiments of the hardware in FIG. 1 of the present invention. In FIG. 6, the system 600 is shown with a multi-well plate design for cells on a shaker/vortex platform, while in FIG. 7, the system 700 is shown with cell culture flasks on a shaker/vortex platform.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A system for cellular analysis comprising:
a computer controller;
one or more syringe pumps;
an incubator comprised of one or more cell cultures;
an autosampler connected to a peristaltic pump, wherein the autosampler may be placed in a refrigerating or incubator system;
a multi-line switch valve in communication with the incubator, said multi-line switch valve configured to add one or more of media, PBS, quenching reagent, or inactivation reagent to the one or more cell cultures, wherein the computer controller mediates the injection of stimulus to the cell cultures via the one or more syringe pumps based on a computed temporal pump profile that determines the stimulus concentration over discrete time points.

2. The system of claim 1, wherein the autosampler collects samples from the incubator at predetermined time points.

3. The system of claim 2, wherein the samples are analyzed by automated cell cytometry.

4. The system of claim 1, further comprising a second peristaltic pump configured to extract waste from the cell culture flasks.

5. The system of claim 1, wherein the cell cultures are plated on multi-well plates.

6. The system of claim 1, wherein the cell cultures are situated in a plurality of cell culture flasks.

7. The system of claim 1, wherein the one or more syringe pumps transmit a flow of cells to the flow cell, in which the cells can be exposed to gradient profiling through the multi-line switch valve.

8. The system of claim 1, wherein a predetermined volume of stimulus is transmitted to the cell cultures over each discrete time point.

9. The system of claim 1, further comprising an air filter in communication with the multi-line switch valve.

10. The system of claim 1, wherein the cell cultures are situated on a stir plate, shaker, or vortex mixer.

11. A method for cellular analysis comprising:
adding one or more of media, PBS, quenching reagent, or inactivation reagent to the one or more cell cultures in an incubator using a multi-line switch valve operated by a computer controller;
mediating the injection of stimulus to the cell cultures via one or more syringe pumps operated by the computer controller based on a computed temporal pump profile that determines the stimulus concentration over discrete time points; and
sampling the one or more cell cultures at predetermined intervals using an autosampler operated by the computer controller.

12. The method of claim 11, further comprising analyzing the samples by automated cell cytometry.

13. The method of claim 11, further comprising operating a second peristaltic pump to dilute the concentration of the stimulus in the cell culture flask and extract waste from the incubator.

14. The method of claim 11, further comprising plating the cell cultures on multi-well plates.

15. The method of claim 11, further comprising situating the one or more cell cultures on a plurality of cell culture flasks.

16. The method of claim 11, further comprising transmitting a flow of cells through the multi-line switch valve for gradient profiling using the one or more syringe pumps.

17. The method of claim 11, further comprising transmitting a predetermined volume of stimulus to the cell cultures over each discrete time point.

18. The method of claim 11, further comprising using an air filter in communication with the multi-line switch valve to remove effluent from the one or more cell cultures.

19. The method of claim 11, further comprising situating the cell cultures on a stir plate, shaker, or vortex mixer.

20. The method of claim 12, further comprising compiling a report of the automated cell cytometry.
